(19) United States
(12) Patent Application Publication
(10) Pub. No.: US 2015/0196515 A1
(43) Pub. Date: Jul. 16, 2015

(54) Multi-Layer Drug Delivery System

(71) Applicant: Dow Corning Corporation, Midland, MI (US)

(72) Inventors: Hyder Aliyar, Midland, MI (US); Robert O. Huber, Midland, MI (US); Gary L. Loubert, Saginaw, MI (US); Gerald K. Schalau, Freeland, MI (US)

(73) Assignee: Dow Coming Corporation, Midland, MI (US)

(21) Appl. No.: 14/419,285

(22) PCT Filed: Mar. 11, 2013
(86) PCT No.: PCT/US13/30226

§ 371 Date: Feb. 3, 2015

Related U.S. Application Data
(60) Provisional application No. 61/683,559, filed on Aug. 15, 2012.

(51) Int. Cl. A61K 31/192; B32B 37/26; B32B 37/18; B32B 37/12; A61K 9/70; A61F 13/02

(52) U.S. Cl. CPC A61K 31/192; A61K 9/7084; A61F 13/0276; B32B 37/18; B32B 37/1284; B32B 37/26; A61F 2013/0296; B32B 2037/268; B32B 2535/00

(57) ABSTRACT

The present disclosure pertains to a construction consisting of in the order from the outside towards the inside: an occlusive or non-occlusive backing substrate; a drug-loaded layer including a therapeutic concentration of at least one or a combination of cosmetic and/or pharmaceutical active ingredients; a permeable or semi-permeable support substrate; a non-drug loaded cured silicone gel adhesive layer cured onto the permeable or semi-permeable support substrate; and a temporary release liner configured to protect the cured silicone gel adhesive layer.

### FIELD OF THE INVENTION

The present disclosure relates to a multi-layer drug delivery system, and, more particularly, to a multi-layer transdermal adhesive patch, wound dressing, or the like for the delivery of active ingredients to and/or through the skin.

### BACKGROUND OF THE INVENTION

Silicone gels, rubbers, and elastomers are the terms generally used to describe elastic materials prepared by the crosslinking of linear polyorganosiloxanes. Gels, elastomers, and rubbers are differentiated by the extent of crosslinking within the siloxane network, by hardness, and elasticity. These materials may be used in medical wound dressings to treat and protect most types of wounds safely. Studies have shown that silicone gel adhesives (SGA) can be removed without causing trauma to the wound or to the surrounding skin or patient. Since silicone is inert, biocompatible, and has good gas permeability, it does not interact chemically with the wound or have any effect upon the cells responsible for the healing process. SGAs may be used for neonatal care, medical device attachment, wound care, skin therapy, scar management and the like.

SGAs do not form a permanent bond. Their soft, rubbery behavior makes such silicone adhesives appropriate materials for contacting biological tissues by minimizing the risk of trauma at the interface. SGAs have properties such as low skin stripping force, gentle removability, and no adhesion to wound bed. Additionally, the skin cells will not lift off when the adhesive is removed, a factor that can damage the skin after repeated removal of traditional acrylic or rubber-based adhesives. Moreover, SGAs do not lose adhesion force when removed from the skin; thus, devices and dressings utilizing such adhesives may be washed with water, air-dried, or reused if necessary. This allows their use in transdermal drug delivery and wound management applications to secure patches or dressings to the skin with minimum impact on the contacting area.

Due to their high permeability, silicones allow the diffusion of many substances such as gases (i.e., oxygen, carbon dioxide, water vapor) but also the diffusion of various actives (i.e., plant extract, drug, or even protein). Thus, silicones are used in personal care, skin topical applications or wound dressings due to their nonocclusive properties. It also explains their use as adhesives or elastomers in controlled drug delivery systems. Moreover, due to their stability, silicones are easy to sterilize by steam or ethylene oxide (EO).

Silicone gel adhesives (SGA) are two-part adhesives that when mixed and fully cured possess the tack properties associated with pressure sensitive adhesives and the resiliency of a soft elastomeric matrix. It has been hypothesized that a therapeutic patch or dressing to deliver an active that employs a SGA as the skin contacting adhesive may be beneficial, because, as a class, the SGAs exhibit no cold flow. These properties, coupled with their ease of removal from skin, have enabled the SGAs to become the adhesive of choice in many advanced wound care and scar care applications. This may also prove a benefit in certain therapies including over the counter (OTC) patches for acute muscle soreness where a patch could be used on multiple areas or for numerous times until it no longer was providing relief.

Silicone pressure sensitive adhesives (PSA) are tacky to the touch, and at the same time they are generally easily removed and do not leave adhesive residue on most substrates. Due to their low liquid surface tension and slightly higher critical surface tension of wetting, silicone adhesives have an excellent ability to flow and wet-out, silicone adhesives conform to the uneven micro-surface of the skin, filling minute gaps and delivering a much broader contact area than traditional adhesives. Silicone adhesives spread easily to form films over substrates like skin and also over their own absorbed film. Moreover, they adhere to the skin securely, forming an immediate bond even on contoured areas of the body. As such, PSAs do not give a gentle adhesion achieved with a SGA, which is beneficial for certain applications such as wound dressing and patches, specifically patches with shorter wear time requirements.

Conventional wound care products incorporate the use of polymeric foams, polymeric films, particulate and fibrous polymers, and/or non-woven and woven fabrics. Dressings with the right combination of these components promote wound healing by providing a moist environment, while removing excess exudate and toxic components, and further serve as a barrier to protect the wound from secondary bacterial infection. The incorporation of various actives to promote wound healing may also be beneficial.

Many of the therapeutic patches on the market today are of the “matrix type,” where the active pharmaceutical as well as any necessary excipients are blended directly into the adhesive, most commonly the adhesive used is a viscoelastic PSA made of silicone, acrylic, rubber or any combination thereof. The adhesive/drug mixture is then devolatized or cured as necessary to create a laminate that can be cut to the appropriate dimensions. Matrix patches using SGA have been prepared, but the actives that can be successfully incorporated into the matrix are limited. It has been found that many drugs including lidocaine, niacinamide, econazole nitrate, ketoprofen, nicotine and salicylic acid either completely inhibited the cure or required special handling and curing procedures in producing the laminate to achieve a cohesive laminate.

There are several medical patches for delivery of active ingredients to the skin available in the art. However, the known patches and dressings do not have the appropriate balance of adhesion force, curing speed, pain management, and flux of the active ingredient into the skin. The embodiments disclosed herein address that need.

### SUMMARY OF THE INVENTION

The present disclosure pertains to a system comprising, in the order from the outside towards the inside: an occlusive or non-occlusive backing substrate; a drug-loaded layer including a therapeutic concentration of at least one or a combination of cosmetic and/or pharmaceutical active ingredients; a permeable or semi-permeable support substrate; a non-drug loaded silicone gel adhesive layer cured onto the permeable or semi-permeable support substrate; and a temporary release liner configured to protect the cured silicone gel adhesive layer.

In one embodiment of the present disclosure, the support substrate may be rate controlling. In another embodiment, the support substrate may be non-rate controlling. The drug-loaded layer may be a non-curing silicone pressure sensitive adhesive. The drug-loaded layer may also be a viscoelastic pressure sensitive adhesive that is an acrylic adhesive or a synthetic rubber pressure sensitive adhesive or any combination thereof.

The present disclosure further comprises a process for the manufacture of a multi-layer drug delivery system comprising: laminating a drug-loaded layer onto a backing substrate; laminating the drug-loaded layer together with the backing substrate onto a first side of a support substrate; a non-drug loaded silicone gel adhesive layer is coated and cured on the second side of the support substrate and applying a temporary release liner onto the non-drug loaded silicone gel adhesive layer.

The present disclosure further comprises another process for the manufacture of a multi-layer drug delivery system comprising laminating a drug-loaded layer onto a backing substrate; coating and curing a non-drug loaded silicone gel adhesive layer onto a first side of a support substrate and covering said non-drug loaded silicone gel adhesive layer with a temporary release liner; and laminating the drug-loaded layer together with the backing substrate onto a second side of the support substrate together with the non-drug loaded silicone gel adhesive layer and the temporary release liner.

The multi-layer delivery system may also include additional optional components. Such optional components may include penetration enhancers, excipients and/or stabilizers. Either the drug-loaded layer, the SGA layer, or both, may contain enhancers known to accelerate the delivery of the active agent through the skin or other substrate. Enhancers are sometimes referred to as skin-penetration enhancers, permeation enhancers, accelerants, adjuvants, and sorption promoters. Either the drug-loaded layer, the SGA layer, or both, may include stabilizers. Stabilizers can include materials that help to prevent drug crystallization, drug degradation, phase separation, etc. and may include polymers, antioxidants, preservatives, surfactants, etc. The drug-loaded layer may also include components traditionally used with or in pressure sensitive adhesives, including crosslinking agents, plasticizers, anti-oxidants, fillers, tackifiers.

### BRIEF DESCRIPTION OF THE DRAWINGS

A more complete appreciation of the present disclosure and the many embodiments thereof will be readily obtained as the same becomes better understood by reference to the following detailed description when considered in conjunction with the accompanying drawing, wherein:

FIG. 1 shows a cross-sectional view schematically illustrating a multi-layer delivery system according to an exemplary embodiment, wherein 101 is a backing substrate, 102 is a drug-loaded layer containing at least one therapeutic active, 103 is a support substrate, 104 is a non-drug loaded SGA layer and 105 is a temporary release liner;

FIG. 2 is a flux profile for ibuprofen based formulation examples 1-4 and a commercial benchmark including ibuprofen;

FIG. 3 is a flux profile for ibuprofen based formulation examples 5, 6, 9, and 10;

FIG. 4 is a flux profile for ibuprofen based formulation examples 5, 7, 8, 11, and 12;

FIG. 5 is a flux profile for estradiol based formulation examples 13, 16, and a commercial benchmark including estradiol;

FIG. 6 is a flux profile for estradiol based formulation examples 13, 18, 19, and a commercial benchmark including estradiol;

FIG. 7 is a flux profile for estradiol based formulation examples 13, 14, 17, and a commercial benchmark including estradiol;

FIG. 8 is a flux profile for estradiol based formulation examples 13, 15, and a commercial benchmark including estradiol;

FIG. 9 is a flux profile for ibuprofen based formulation examples 20-23.

### DETAILED DESCRIPTION

Features and advantages of the present disclosure will now be described with occasional reference to specific embodiments. However, the invention may be embodied in different forms and should not be construed as limited to the embodiments set forth hereby. Rather, these embodiments are provided so that this disclosure will be thorough and complete and will fully convey the scope of the disclosure to those skilled in the art.

Unless otherwise indicated or defined, all technical and scientific terms used herein have the same meaning as commonly understood by one of ordinary skill in the art to which the invention pertains. The terminology used herein is for describing particular embodiments only and is not intended to be limiting. The term “ambient conditions” as used throughout the specification refers to surrounding conditions under about one atmosphere of pressure, at about 50 percent relative humidity, and at about 25° C., unless otherwise specified.

Unless otherwise indicated, all numbers expressing quantities of ingredients, properties such as molecular weight, percent by weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present disclosure. Notwithstanding that the numerical rangers and parameters setting forth the broad scope of the disclosure are approximations, the numerical values set forth in the specific examples are reported as precisely as possible. Any numerical values, however, inherently contain errors necessarily resulting from error found in their respective measurements.

All percentages, parts, and ratios are based upon the total weight of the formulation, unless otherwise specified. All such weights as they pertain to listed ingredients are based on the active level and, therefore, do not include carriers or by-products that may be included in commercially available materials, unless otherwise specified.

According to one embodiment, the multi-layer delivery system according to the present disclosure is a patch that may be applied to a substrate which is typically a biological surface, human body tissue, and/or animal body tissue. More specific substrates include, but are not limited to, skin, mucous membrane, and nails.

The multi-layer delivery system constructed according to the present disclosure is typically applied for topical therapy, such as to treat damaged or diseased skin, and wound care, such as to treat cuts, burns, scars, and the like. The present disclosure, including multi-layer patches constructed according to the disclosure, may also be applied in various transdermal, pharmaceutical, veterinary, and oral health care applications.

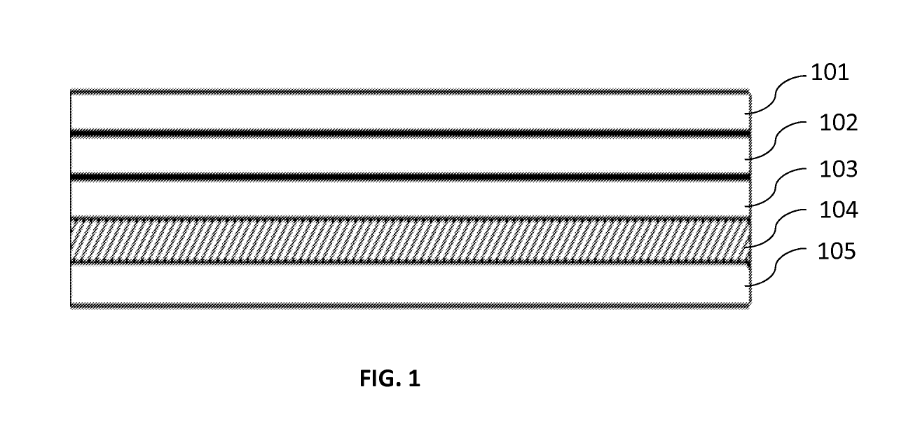

Referring now to FIG. 1, described herein is a system comprising, in the order from the outside toward the inside, an external occlusive or non-occlusive backing substrate layer 101; a drug-loaded layer 102 including a therapeutic concentration of at least one or a combination of cosmetic and/or pharmaceutical active ingredients; a permeable or semi-permeable support substrate 103; a non-drug loaded silicone gel adhesive layer 104 cured onto a permeable or semi-permeable support substrate 103; and a temporary release liner 105 adapted to cover and/or protect the silicone gel adhesive layer 104.

As used herein, a “silicone gel” or SGA is an elastic, tacky, jelly-like solid material formed by lightly cross-linking silicone polymers. By contrast, pressure sensitive adhesives (PSA) have low, negligible, or no resiliency.

### Backing Substrate (101)

The first element in the system is an external backing substrate 101. The primary purpose of the backing substrate 101 is to form a layer over the drug-loaded layer 102 to prevent the system from adhering via the drug-loaded layer 102 to clothing or other objects that the system may come into contact with during use. The backing substrate 101 also contributes to wear strength, tensile strength, and tear strength of the construction. Backing substrate layers produced from silicone, fabric, coated papers, aluminum, plastics including polyesters, polyurethanes, or the like, or any combination thereof are useful in systems and processes according to the present disclosure. The backing substrate 101 may have a thickness of less than about 0.15 mm. The backing substrate 101 may have a thickness of between about 0.012 mm to about 0.1 mm. Alternatively, the backing substrate 101 may also have any thickness that is suitable for the particular application for which the multi-layer system is used.

The backing substrate may be occlusive. According to another embodiment, the backing substrate may be non-occlusive. Occlusive is generally understood to mean that the backing substrate may be one that prevents the transepidermal water loss which is believed to increase the permeability capability of the skin.

Drug-loaded layer (102)

The drug-loaded layer 102 may be adhered to one side of the backing substrate 101, such that the drug-loaded layer is positioned between and in contact with the backing substrate 101 and the support substrate 103 and the SGA layer 104 is positioned between and in contact with the support substrate 103 and the temporary release liner 105 as shown in FIG.

The drug-loaded layer 102 may be a non-curing silicone pressure sensitive adhesive (PSA). Alternatively, the drug-loaded layer 102 may be a viscoelastic PSA that is an acrylic adhesive, a silicone-acrylic hybrid adhesive or a synthetic rubber pressure sensitive adhesive.

The drug-loaded layer 102 can include any viscoelastic material which adheres to most substrates with application of slight pressure and essentially remains tacky through the useful life of the construction. The drug-loaded layer 102 may be silicone, polyisobutylene and derivatives thereof, acrylics, natural rubbers, natural and synthetic polyisoprene, polybutylene and polyisobutylene, styrene/butadiene polymers, styrene-isoprene-styrene block polymers, hydrocarbon polymers such as butyl rubber, halogen polyvinylchloride, polyvinylidene chloride, polyvinylpyrrolidone and polychlorodiene and combinations thereof. By non-curing it is meant that the adhesive layer 102 is thermoplastic and is applied by hot-melt or a solvent based process and typically does not undergo further curing to solidify.

Silicone PSAs useful hereby generally comprise the product of (I) a silanol-terminated polydiorganosiloxane crosslinked with (II) a silanol-containing silicone resin. The silanol-terminated polydiorganosiloxane may have a viscosity of about 0.1 Pa.s to about 30000 Pa.s, alternatively about 1 Pa.s to about 100 Pa.s at 25° C. Methods for producing silanol-terminated polydiorganosiloxane are well known in the art.

The silanol-containing silicone resin (II) is typically a non-linear siloxane resin and consists of siloxane units of the formula R¹ₐSiO_{(4-a)/2} where R¹ is a hydroxyl, hydrocarbon or hydrocarbonoxy group and a has an average value of from 1 to 1.8. The resin may be comprised of groups having the formula R²₃SiO_{1/2}(“M” groups) and groups having the formula SiO_{4/2} (“Q” groups) where R² is an alkyl group having 1 carbon to 6 carbon atoms. R² may be methyl. The number ratio of M groups to Q groups may be in the range of about 0.05:1 to about 1.2:1 (equivalent to the value of a in the formula R¹ₐSiO_{(4-a)/2} being between about 1.0 and about 1.63), alternatively, the number ratio of M groups to Q groups may be in the range of about 0.6:1 to about 0.9:1. The silicone resin may contain at least about 0.2 percent by weight to about 5 percent by weight, or, alternatively about 0.5 percent by weight to about 3 percent by weight of silicone-bonded hydroxyl groups. These silicone-bonded hydroxyl groups may be present as M groups: (OH)(CH₃)₂SiO_{1/2}.

The silicone PSA may be produced from about 20 parts to about 80 parts by weight, alternatively about 30 parts to about 60 parts by weight of the silanol-terminated polydiorganosiloxane and about 80 parts to about 20 parts by weight, or, alternatively, about 70 parts to about 40 parts by weight of the silanol-containing silicone resin. Alternatively, the silicone PSA may be produced from about 30 parts to about 60 parts by weight of a silanol-terminated polydiorganosiloxane having a viscosity of about 0.1 Pa.s to about 30000 Pa.s at 25° C. and about 40 parts to about 70 parts by weight of a silanol-containing silicone resin having M and Q groups as defined above, with the number ratio of M to Q in the range of about 0.5:1 to about 1.2:1.

To produce the silicone PSA, the silanol-terminated polydiorganosiloxane and silanol-containing silicone resin are typically mixed together. The silanol groups of the polydiorganosiloxane generally undergo some condensation with the silanol groups of the silicone resin such that the polydiorganosiloxane is crosslinked with the resin. A catalyst, for example an alkaline material, can be added to promote the crosslinking reaction. Useful catalysts additionally include ammonia, ammonium hydroxide, ammonium carbonate, and other suitable catalysts.

Remaining silanol groups on the silicone PSA produced above may be at least partially reacted with an end-blocking agent that introduces a triorganosilyl unit. The endblocking agent can be exemplified by disilazanes such as hexamethyldisilazane or a trialkyl alkoxy silane such as trimethyl ethoxy silane or trimethyl methoxy silane. Methods for end-blocking silanol containing silicone PSAs are known in the art.

Silicone adhesives produced from a silanol-terminated polydiorganosiloxane and an acetoxysilane may also be used here. These adhesives are commercially available from and sold by Dow Corning Corporation (Midland, Mich.).

Another type of silicone PSA that can be used hereby are those silicone PSA described in U.S. Patent Publication No. 2008/0138386, which is hereby incorporated by reference in its entirety. In particular, these materials are a cross-linkable composition comprising a saccharide-siloxane copolymer, a crosslinking agent and, optionally, a solvent.

Another type of silicone PSA that can be used herein are those silicone PSA described in U.S. patent application Ser. No. 12/203,362, which is hereby incorporated by reference in its entirety. In particular, this PSA is a reaction product of a silicone-containing PSA, an ethylenically unsaturated monomer and an initiator. The silicone containing PSA may be produced from (I) a silanol-terminated polydiorganosiloxane crosslinked with (II) a silanol-containing silicone resin as described above. The ethylenically unsaturated monomer can be any monomer having at least one carbon-carbon double bond. The ethylenically unsaturated monomer may be a compound selected from aliphatic acrylates, aliphatic methacrylates, cycloaliphatic acrylates, cycloaliphatic methacrylates, and combinations thereof. Initiators may include peroxides, azo-compounds, redox initiators, and photo-initiators.

According to one embodiment of the present disclosure, the drug-loaded layer 102 does not include any penetration enhancers. According to an alternate embodiment, the drug-loaded layer 102 may include between about 0.5 to about 25 percent by weight of one or more penetration enhancers. According to an alternate embodiment, the drug-loaded layer 102 may include between about 1 to about 15 percent by weight of one or more penetration enhancers.

The drug-loaded layer 102 may be free of stabilizers. According to an alternate embodiment, the drug-loaded layer 102 may include between about 0.01 to about 25 percent by weight of one or more stabilizers. The drug-loaded layer 102 may include between about 0.1 to about 15 percent by weight of one or more stabilizers.

The drug-loaded layer 102 may have a thickness of from about 0.0005 mm to about 1 mm. The drug-loaded layer 102 may have a thickness of from about 0.0125 mm to about 0.25 mm.

### Therapeutic Active

The multi-layer system is particularly suitable for delivery of actives that cannot be delivered with a SGA alone because they interfere with the curing of the components used to produce the SGA or if incorporating them into the SGA would destroy the structure of the gel. In particular, actives that contain a functional group selected from amine, sulfur, nitrogen-heterocyclic, acetylenic, unsaturated hydrocarbon monoester and diester, conjugated ene-yne, hydroperoxide, nitrile and diaziridine are useful for delivery by the multi-layer system. Specific actives and classes of actives that can be delivered by the construction include vitamins, including tocopherol (vitamin E) and vitamin A palmitate, lidocaine, salicylic acid, econazole nitrate, levongestrel, niacinamide, nicotine, ketoconazole, azole, steroids, retinoids, and non-steroidal anti-inflammatory drugs.

Functional molecules that may inhibit the cure of platinum catalyzed Si—H to vinyl addition reactions, include nitrogen containing compounds like amines and amides, nitriles, cyanates, oximo, nitroso, hydrazo, azo compounds, and nitrogen chelates, sulfur containing compounds such as sulfides and thio compounds, phosphorus containing compounds including phosphines, phosphites and phosphates, and organic compound such as alcohols, ketones, aldehydes, carboxylic acids, esters, and organic molecules with unsaturated bonds.

The amount of the active or combination of actives incorporated into the drug-loaded layer 102 varies depending on a variety of factors including, but not limited to, the particular active agent, the desired therapeutic effect, and the time span for which the system is to provide therapy. For most active agents, the rate of passage of the active agent through the skin (the flux) is the rate-limiting step in transdermal delivery. The amount of active agent and the rate of release are typically selected so as to provide a transdermal delivery characterized by a zero order time dependency for a prolonged period of time. The minimum amount of active agent in the PSA is selected based on the active agent which passes through the skin, or other substrate, in the time span for which the construction is to provide therapy.

The amount of active agent in the drug-loaded layer 102 may be between about 0.1 percent by weight to about 80 percent by weight, based on the weight of the drug-loaded layer 102. Alternatively, the amount of active may be from about 0.3 percent by weight to about 50 percent by weight, or, between about 1.0 percent by weight and about 30.0 percent by weight, based on the weight of the drug-loaded layer 102.

### Enhancers

The drug-loaded layer 102 and/or the silicone gel adhesive layer 104 may contain enhancers. Enhancers are sometimes referred to as skin-penetration enhancers, permeation enhancers, accelerants, adjuvants, and sorption promoters. Enhancers include those with diverse mechanisms of action including those which have the function of improving the solubility and diffusibility of the active agent within the composition and those which improve percutaneous absorption, for example, by softening the skin, improving the skin's permeability, acting as penetration assistants or hair follicle openers or changing the state of the skin including the boundary layer. Some of these enhancers have more than one mechanism of action, but in essence they serve to enhance the delivery of the active agent to or through the substrate.

The enhancers may be exemplified by, but not limited to, polyhydric alcohols such as dipropylene glycol, propylene glycol and polyethylene glycol which enhance solubility of the active agent; oils such as olive oil, squalene, and lanolin; fatty ethers such as cetyl ether and oleyl ether; fatty acid esters such as isopropyl myristate which enhance diffusibility of the active agent; urea and urea derivatives such as allantoin which affect the ability of keratin to retain moisture; polar solvents such as dimethyldecylphophoxide, methyloctylsufoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the substrate (skin) and the active agents administrated. Other enhancers include oleic and linoleic acids, ascorbic acid, panthenol, butylated hydroxytoluene, tocopherol, tocopheryl acetate, tocopheryl linoleate, propyl oleate and isopropyl palmitate.

### Stabilizers

The system according to the present disclosure may include stabilizers. Stabilizers may include crystallization inhibiting agents. One known agent is polyvinylpyrrolidone (PVP). The term PVP refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum soluble, and Poly(1-vinyl-2-pyrrolidone). The copolymer vinyl acetate vinylpyrrolidone is known to the pharmaceutical industry as Copolyvidon, Copolyvidone, and Copolyvidonum. The term “soluble” when used with reference to PVP means that the polymer is soluble in water and generally is not substantially cross-linked, and has a molecular weight of less than about 2,000,000.

Stabilizers according to the present disclosure may also include preservatives including antioxidants. Illustrative examples of suitable preservatives include alpha tocopherol, ascorbic acid, ascorbyl palmitate, butylated hydroxyanisole, butylated hydroxytoluene, ethyl oleate, fumaric acid, malic acid, monothioglycerol, phosphoric acid, potassium metabisulfite, propionic acid, propyl gallate, sodium ascorbate, sodium metobisulfite, sodium sulfite, citric acid monohydrate, tartaric acid, tocopherol, vitamin E, benzalkonium chloride, benzethonium chloride, benzoic acid, benzyl alcohol, bronopol, butylparaben, cetrimide, chlorbutanol, chlorhexidine, chlorobutanol, chlorocresol, chloroxylenol, cresol, ethylparaben, glycerin, hexetidine, imidurea, lactic acid, methylparaben, phenol, phenoxyethanol, phenylethyl alcohol, potassium benzoate, potassium sorbate, propylparaben, xylitol, acacia, aalbumin, alginic acid, calcium stearate, carboxymethylcellulose calcium, carboxymethylcellulose sodium, colloidal silcon dioxide, cyclodextrins, diethanolamine, edentates, ethylcellulose, ethylene glycol palmitostearate, glycerin monostearate, guar gum, hydroxypropyl cellulose, hypromellose, lecithin, magnesium aluminum silicate, monoethanolamine, polacrilin potassium, polyoxamer, polyvinyl alcohol, potassium chloride, povidone, propylene glycol, propylene glycol alginate, propyl gallate, sodium alginate, sodium stearyl fumarate, sorbitol, stearyl alcohol, trehalose, white wax, xantham gum, yellow wax.

### Support Substrate (103)

The support substrate 103 is configured to withstand curing of the adhesive layer 104 at elevated temperatures. Curing temperatures may be from about 40° C. to about 150° C. The support substrate 103 may be constructed of a permeable or a semi-permeable material onto which the cured silicone adhesive layer 104 is coated and cured.

The support substrate material 103 is selected such that the active ingredient included in the drug-loaded layer 102 can permeate or penetrate through it. Therefore, the support substrate 103 may not be occlusive. The support substrate 103 may be fabricated from permeable, semipermeable, or microporous materials which may or may not control the rate of active agents and/or fluids into and out of delivery devices. The support substrate 103 materials can include a wide variety of materials including paper, natural or synthetic fibers, woven cloth, nonwovens, plastic films and/or foams made from materials such as cotton, rayon, wool, hemp, jute, nylon, polyesters, polyacetates, polyacrylics, alginates, ethylene-propylene-diene rubbers, natural rubber, silicones, polyesters, polyisobutylenes, polyolefins (e.g., polypropylene polyethylene, ethylene propylene copolymers, and ethylene butylene copolymers), polyurethanes, vinyls including polyvinylchloride and ethylene-vinyl acetate, polyamides, polystyrenes, fiberglass, ceramic fibers, and/or any combinations thereof.

The support substrate may have a thickness of between about 0.010 mm and about 1 mm. The drug-loaded layer 102 may be coated on an outer surface of the support substrate 103 and the silicone gel adhesive layer 104 may be laminated on an inner surface of the support substrate 103 as shown in FIG. 1

### Non-Drug Loaded Silicone Gel Adhesive Layer (104)

The non-drug loaded SGA layer 104 may include a silicone resin-reinforced SGA that includes between about 2 to about 45 percent by weight of at least one hydroxyl substituted siloxane resin.

The non-drug loaded SGA layer 104 may be free of penetration enhancers. In an alternative embodiment, the non-drug loaded cured SGA layer 104 may include between about 0.5 to about 25 percent by weight of one or more penetration enhancers. In an alternative embodiment, the non-drug cured SGA layer 104 may include between about 1 to about 15 percent by weight of one or more penetration enhancers.

The non-drug loaded SGA layer 104 may be flood coated and cured onto the support substrate 103. Flood coating refers to coating of the entire surface of the support substrate 103 by the non-drug loaded SGA layer 104. Flood coating may be used to achieve a desired texture or to create added dimension.

Alternatively, the SGA layer 104 may be pattern coated and cured onto the support substrate 103. Properties of the SGA layer 104 including viscosity, shear thinning behavior, adhesiveness, and cohesive strength are important for pattern coating the silicone gel adhesive layer 104 onto the support substrate 103. A desirable combination of properties exhibited by the silicone gel adhesive layer 104, including the adhesion, viscosity, cohesive strength, and rheology may enable for the silicone gel adhesive layer 104 to be pattern coated onto the support substrate 103. The SGA layer 104 may exhibit (i) viscosity ranging from about 7000 cP to about 5,000,000 cP and (ii) shear thinning behavior, as determined by the rheological profile. Once the silicone composition is pattern coated onto a substrate, the pattern of the coating is able to be maintained upon application. The SGA layer 104 exhibits (i) adhesiveness ranging from about 0.2 N to about 4 N and (ii) cohesive strength, as determined by the peel adhesion test.

Applying the SGA layer 104 onto the support substrate 103 in a pattern naturally creates discontinuity in the areas on the support substrate 103 that are not coated with the silicone composition. Similar to creating a carrier material with perforations, applying the discontinuous (or semi-continuous) pattern on the support substrate 103 creates a coating with void areas that allow exudate to pass through to the substrate to be absorbed in a wound dressing application. Any predetermined pattern that creates the void areas is sufficiently discontinuous for these purposes. The discontinuity of the pattern also enables an avenue for the moisture to be released from the wound, promoting a balanced moisture vapor. However, in a patch application, flood coating may be preferred as the increased skin contact area is beneficial for drug delivery.

The curing of the SGA layer 104 onto the support substrate 103 may occur at an elevated temperature of between about 40° C. to about 150° C. Alternatively, the curing of the SGA layer 104 onto the support substrate 103 may occur at an elevated temperature of between about 60° C. to about 120° C. The curing temperature may be adjusted depending on the therapeutic active, the specific properties of the silicone gel adhesive, the composition and thermal stability of the support substrate or other factors.

The SGA layer 104 may saturate the support substrate 103. Alternatively, the SGA layer 104 may not saturate the support substrate 103. If the SGA layer 104 saturates the adhesive layer, that means that the SGA layer 104 soaks the support substrate 103 while it is curing.

The non-drug loaded SGA layer 104 may have a thickness of about 0.05 mm to about 2 mm. Alternatively, the non-drug loaded SGA layer 104 may have a thickness of from about 0.1 mm to about 1 mm.

The SGA used in the present disclosure should be chosen to have the properties desired for the end application. Important properties may include softness, friability and strength.

The SGA used in the present disclosure are generally formed from linear or branched silicones having reactive functional groups thereon. Such reactive groups undergo a cross-linking reaction during curing. Examples of cross-linking reactions include the hydrosilylation reaction in which a silicone having an Si—H reactive group reacts with a silicone having an aliphatic unsaturated reactive group in the presence of a hydrosilylation catalyst. An alternative reaction is the condensation cure in which an alkoxy and/or hydroxy containing siloxanes are cured with a catalyst as described in U.S. Pat. No. 4,831,070, which is hereby incorporated by reference in its entirety.

Typically, the silicone gels are obtained by reacting a gel producing composition comprising (A) at least one alkenyl-substituted polydiorganosiloxane, (B) at least one organosiloxane containing silicone-bonded hydrogen atoms, and (C) at least one catalyst for the reaction of the SiH groups with the Si-alkenyl groups. These compositions cure at normal ambient temperatures, but curing can be expedited by heating to elevated temperatures, such as temperatures from about 40° C. to about 150° C.

The alkenyl-substituted polydiorganosiloxanes (A) are known in the art. Suitable alkenyl groups contain from 2 carbon atoms to about 6 carbon atoms and are exemplified by, but not limited to, vinyl, allyl, and hexenyl. The alkenyl groups in this component may be located at terminal, pendant (non-terminal), or both terminal and pendant positions. The remaining silicone-bonded organic groups in the alkenyl-substituted polydiorganosiloxane are independently selected from monovalent hydrocarbon and monovalent halogenated hydrocarbon groups free of aliphatic unsaturation. These groups may contain from 1 carbon to about 20 carbon atoms, or, alternatively, from 1 carbon to 8 carbon atoms. These silicone-bonded organic groups are exemplified by, but not limited to, alkyl groups such as methyl, ethyl, propyl, and butyl; aryl groups such as phenyl; and halogenated alkyl groups such as 3,3,3-trifluoropropyl. In some embodiments, at least about 50 percent of the silicone-bonded organic groups in the alkenyl-substituted polydiorganosiloxane are methyl.

The structure of the alkenyl-substituted polydiorganosiloxane is typically linear however; it may contain some branching due to the presence of trifunctional siloxane units. The viscosity of the alkenyl-substituted polydiorganosiloxane can be selected according to the application, as needed. For example, it can be between greater than 0 mm²/s to about 100,000 mm²/s, alternatively, between about 50 mm²/s to about 80,000 mm²/s, or, alternatively, between about 300 mm²/s to about 3,000 mm²/s.

Methods for preparing the alkenyl-substituted polydiorganosiloxanes of the present invention, such as condensation of the corresponding halosilanes or equilibration of cyclic polydiorganosiloxanes, are well known in the art.

The alkenyl-substituted polydiorganosiloxanes can be used in the gel producing composition in an amount of between about 10 percent by weight to about 90 percent by weight based on the weight of the composition. Alternatively, the alkenyl-substituted polydiorganosiloxanes can be used in an amount between about 40 percent by weight to about 90 percent by weight, or, alternatively, in an amount between about 50 percent by weight to about 80 percent by weight. The amount of alkenyl groups present in the alkenyl-substituted polydiorganosiloxane may be in the range of between about 0.05 percent by weight to about 1 percent by weight based on the weight of the alkenyl-substituted polydiorganosiloxane.

The organosiloxanes containing silicone-bonded hydrogen atoms (B) are also known in the art. The hydrogen atoms in this component may be located at terminal, pendant (non-terminal), or both terminal and pendant positions. The remaining silicone-bonded organic groups in this component are independently selected from monovalent hydrocarbon and monovalent halogenated hydrocarbon groups free of aliphatic unsaturation. These groups typically include from 1 carbon to about 20 carbon atoms, alternatively from 1 carbon atom to 8 carbon atoms, and are exemplified by, but not limited to, alkyls such as methyl, ethyl, propyl, and butyl; aryls such as phenyl; and halogenated alkyls such as 3,3,3-trifluoropropyl. In one embodiment of the disclosure, at least about 50 percent of the organic groups in the organosiloxane containing silicone-bonded hydrogen atoms are methyl.

The structure of the organosiloxane containing silicone-bonded hydrogen atoms is typically linear however; it may contain some branching due to the presence of trifunctional siloxane units. The viscosity of the organosiloxane containing silicone-bonded hydrogen atoms can be selected depending on the application, as needed. For example, it can be greater than 0 mm²/s to about 100,000 mm²/s, alternatively, about 5 mm²/s to about 500 mm²/s.

The organosiloxanes containing silicone-bonded hydrogen atoms can be used in the gel producing composition in an amount of about 1 percent by weight to about 30 percent by weight based on the weight of the composition, alternatively about 5 percent by weight to about 20 percent by weight, and, alternatively, about 5 percent by weight to about 15 percent by weight. In one embodiment, the amount of hydrogen groups present in the organosiloxane containing silicone-bonded hydrogen atoms is between about 0.05 percent by weight to about 1.44 percent by weight based on the weight of the organosiloxane containing silicone-bonded hydrogen atoms.

In the gel producing compositions, (A) and (B) are selected such that the ratio of hydrogen (as SiH) to Alkenyl (as Si-Alkenyl) is generally in the range of about 0.1:1 to about 10:1.

The hydrosilylation catalyst promotes the addition reaction of the alkenyl- substituted polydiorganosiloxane with the organosiloxane containing silicone-bonded hydrogen. The hydrosilylation catalyst can be any of the well-known hydrosilylation catalysts comprising a platinum group metal, a compound containing a platinum group metal, or a microencapsulated platinum group metal or compound containing the same. These platinum group metals include platinum, rhodium, ruthenium, palladium, osmium and iridium. Platinum and platinum compounds are selected based on their high activity level in hydrosilylation reactions. One class of platinum catalysts includes the complexes of chloroplatinic acid with certain vinyl-containing organosiloxane compounds disclosed in U.S. Pat. No. 3,419,593, which is hereby incorporated by reference in its entirety. A specific catalyst of this type is the reaction product of chloroplatinic acid and 1,3-diethenyl-1,1,3,3-tetramethyldisiloxane.

The hydrosilylation catalyst is present in an amount sufficient to cure the composition of the present disclosure. Typically, the concentration of the catalyst sufficient in the systems and processes according to the present disclosure is from about 0.1 ppm to about 500 ppm (parts per million), alternatively from about 1 ppm to about 100 ppm, alternatively from about 1 ppm to about 50 ppm of a platinum group metal, based on the weight of (A) and (B).

If desired, other components can be included in the gels of the present invention including, but not limited to, fillers, pigments, low temperature cure inhibitors, additives for improving adhesion, cross-linkers (e.g., Si—H cross-linkers), chain extenders, pharmaceutical agents, cosmetic agents, natural extracts, fluids or other materials conventionally used in gels. Other optional components include silicone fluids, silicone waxes, silicone polyethers, and other polymers including, for example, hydrophilic polymers such as sodium polyacrylic acid, PVA, PVP, polyacrylic adhesive, cellulose and polysaccharide (which can make the gel more hydrophilic and more permeable to moisture). Still other optional components include rheology modifiers such as thickening agents, thixotropic agents and materials that react with the ingredients in the gel such as castor oil or maleates that can react with the hydroxyl groups of the resin. In some embodiments, the gel contains substantially no fillers (e.g., less than about 5 percent by weight, or, alternatively, less than about 1 percent by weight, or, alternatively, less than about 0.1 percent by weight). In some embodiments, the gel contains substantially no solvent (e.g., less than about 5 percent by weight, or, alternatively less than about 1 percent by weight, or, alternatively, less than about 0.1 percent by weight).

Another optional ingredient that may be included in the silicone gel is a hydroxy substituted silicone resin as described in U.S. Patent App. No. 2007-0202245, hereby incorporated by reference in its entirety. The resin is typically comprised of groups having the formula R³₃SiO_{1/2} (“M” groups) and groups having the formula SiO_{4/2} (“Q” groups), where R³ is an alkyl group having 1 carbon to 6 carbon atoms or an alkylene group having 1 carbon to 6 carbon atoms, which in some embodiments may be methyl or vinyl.

If an alkenyl group is present in the resin, typically the mole percentage of R groups present as alkenyl groups is less than about 10 mole percent, or, alternatively, less than about 5 mole percent.

The number ratio of M groups to Q groups may be in the range of about 0.6:1 to about 4:1, or, alternatively about 0.6:1 to about 1.0:1. The silicone resin may contain between about 0.1 percent by weight to about 5 percent by weight, or, alternatively, about 1.0 percent by weight to about 5 percent by weight of silicone-bonded hydroxy groups.

The resin may be used in the gel producing composition in an amount of between about 2 percent by weight to about 45 percent by weight, based on the weight of the gel producing composition and resin, or, alternatively, in an amount between about 5 percent by weight to about 45 percent by weight, or, alternatively, in an amount between about 10 percent by weight to about 35 percent by weight.

The SGA layer 104 may be made by processes known in the art. For example, the gel may be preformed (e.g., as a sheet) by molding, calendaring, extruding, spraying, brushing, applying by hand, casting or coating the silicone gel adhesive layer on a substrate such as a temporary release liner 105 or the support substrate 103.

Alternatively, the SGA layer 104 may be made by applying the gel producing composition to a substrate such as a temporary release liner 105 or the support substrate 103 by spraying, coating, bar coating, and other known methods. Once applied to the substrate, the gel producing composition is cured to produce the silicone gel adhesive on the substrate.

The silicone gel adhesives useful herein may have a penetration of about 5 mm to about 300 mm, alternatively, between about 50 mm to about 300 mm, as determined by the cone penetration test method based on ASTM D-217-88 using a cone category 1806-1 weighted 62.5 g.

The adhesive strength of the silicone gel adhesive should be sufficient to maintain adhesion to the substrate to which it is applied (i.e. skin), yet not too strong that it would damage the substrate upon removal. The tack of the SGA, when measured with a probe tack tester, may be between about 50 g to about 500 g, or, alternatively between about 150 g to about 350 g.

Depending on the active to be delivered, the SGA layer 104 may or may not act as a rate controlling layer for the diffusion of the active.

### Temporary Release Liner (105)

In the construction, the drug-loaded layer 102 is covered on an outer side by the backing substrate 101 and by the support substrate 104 on an inner side. The support substrate 103 is covered on the inner side by the silicone gel adhesive layer 104 as seen in FIG. 1. After the construction is prepared, one side of the silicone gel adhesive layer 104 is exposed. This side will be the side that contacts the surface such as human skin. If desired, this surface of the silicone gel adhesive layer 104 may be covered or protected with a temporary release liner 105 prior to use. Suitable release liner 105 materials are known in the art and can include a plastic, a silicone, a fluorinated silicone, a fluorine polymer, polyethylene, perfluoro based polymers, perfluoropolyether based polymer, PVC, and others. Additionally, the release liner 105 could be made from a wide variety of materials such as paper coated with a suitable release coating. The surface of the release coating can be smooth, embossed or it may have any other desirable form.

### System Construction

The system may be manufactured in a variety of ways to obtain the system shown in FIG. 1. One process for manufacturing the system includes producing a blend of the drug-loaded layer 102, the active ingredient and other additives as needed and forming a layer of the blend onto the inner surface of the backing substrate 101. A layer of an uncured SGA 104 is then applied on a first side of the support substrate 103, the SGA is cured and covered with a temporary release liner 105. The drug-loaded layer together with the backing substrate is then laminated to a second side of the support substrate 103 together with the SGA layer 104 and temporary release liner 105. The curing of the silicone gel adhesive may be done before or after coating or laminating the blend on an outer side of the support substrate 103.

The silicone gel adhesive layer 104 may be pattern coated and cured onto the substrate 103. Alternatively, the silicone gel adhesive 104 may be flood coated and cured onto the substrate 103.

Typically the layers 102, 103, and 104 are held together by their natural affinity. However, it may be desirable to apply pressure once the surfaces are brought into contact to ensure bonding between the materials.

Other additives or agents commonly added to medical dressings may also be included in the system. For instance, the multi-layer system may also include agents that provide a pain-relieving effect, antiseptic effect, help sterility, and speed healing. The agents may be added separately or impregnated into the silicone composition, absorbable substrate, or other component of the medical dressing. For instance, multi-layer systems are commonly impregnated with antiseptic chemicals, such as in boracic lint. In one embodiment, the multi-layer system may include silver particles, either suspended in the adhesive gel or otherwise impregnated into the dressing, which can be used to impart antimicrobial properties into the dressing.

The system prepared according to the present disclosure is configured to provide an increased flux through the skin compared to the flux observed using the drug-loaded drug-loaded layer 102 together with a backing substrate 101. The system may be configured to deliver between about 0.1 percent to about 80 percent of the active ingredient to the skin. The percent active delivered may be adjusted by one of ordinary skill in the art depending on the demands of the particular application.

### EXAMPLES

These examples are intended to illustrate the invention to one of ordinary skill in the art and should not be interpreted as limiting the scope of the invention set forth in the claims. All measurements and experiments were conducted at 25° C., unless indicated otherwise. Each formulation example was tested in triplicate,

As used herein,

Ibuprofen, USP grade, available from Spectrum Chemical Mfg. Corp., Gardena, Calif.;

Estradiol, USP grade, available from Spectrum Chemical Mfg. Corp., Gardena, Calif.;

Benchmark 1 is Ibutop (5% Ibuprofen gel), available from Dolorgiet, Bonn, Germany;

Benchmark 2 is VIVELLEDOT® (0.156 mg/cm² estradiol), available from Novartis Pharmaceuticals, East Hanover, N.J.;

K90 is KOLLIDON® K90, a polyvinylpyrrolidone, available from BASF, Ludwigshafen, Germany;

K25 is KOLLIDON® K25, a polyvinylpyrrolidone, available from BASF, Ludwigshafen, Germany;

VA64 is KOLLIDON® VA64, a vinylpyrrolidone vinyl acetate-copolymer, available from BASF, Ludwigshafen, Germany;

Ethyl Acetate, HPLC grade, available from Acros, Fair Lawn, N.J.;

2-propanol, HPLC grade, available from Fisher Scientific, Fair Lawn, N.J.;

Dipropylene glycol, available from Spectrum Chemical Mfg. Corp., Gardena, Calif.;

Oleic Acid, NF/FCC grade, available from Fisher Scientific, Fair Lawn, N.J.;

Isopropyl tetradecanoate, 98%, available from Alfa Aesar, Heysham, Lancashire, UK;

SCOTCHPAK® 1022 is a fluoropolymer coated polyester film, available from 3M, St. Paul, Minn.;

SCOTCHPAK® 8038 is a polyester film, available from 3M, St. Paul, Minn.;

SGA-1 isan experimental SGA, Dow Corning, Midland, Mich.;

SGA-1 is a two part curing adhesive including a part A produced by blending 99.72 wt. % vinyl-terminated polydimethylsiloxane with 0.280 wt. % platinum complex and a part B produced by blending 98.78 wt. % vinyl-terminated polydimethylsiloxane, 1.19 wt. % hydrogen-functional polydimethylsiloxane and 0.030 wt. % inhibitor.;

SGA-2 is a two-part high adhesion elastomeric silicone adhesive, available from Dow Corning, Midland, Mich.;

PSA-1 is a pressure sensitive silicone adhesive in solvent, available from Dow Corning, Midland, Mich.;

PSA-2—is a pressure sensitive developmental adhesive, available from Dow Corning, Midland, Mich.;

SS-1 is a nonwoven polypropylene, available from of Polymer Science Inc., Monticello, Ind.;

SS-2 is a silicone elastomer membrane, available from Dow Corning, Midland, Mich.;

SS-3 is a Celgard 2500 Microporous Membrane, available from Celgard, Charlotte, N.C.;

LDPE is a low-ensity polyethylene, available from Bloomer Plastics Inc., Bloomer, Wis.

### Example 1

The drug-in-adhesive layer was prepared by dispersing 0.8015 g ibuprofen and 0.9026 g K90 into 1.5118 g ethyl acetate. To the dispersion, 1.5141 g 2-propanol was added to dissolve the ibuprofen and the K90. To this solution, 0.3159 g dipropylene glycol/oleic acid (2:1) blend was added. The mixture was mixed overnight in a vial and coated onto SCOTCHPAK® 1022 using an 8 mil (203.2 um) applicator bar. The laminate was dried for 5 minutes at ambient conditions, followed by 5 more minutes at 92° C. The adhesive was laminated to SCOTCHPAK® 8038. The resulting coat weight was approximately 5.22 mg/cm² with an ibuprofen content of 2.1 mg/cm² (0.495 cm² patch-1.04 mg ibuprofen available).

### Example 2

To produce the skin-contact layer, equal parts A and B of SGA-1 were mixed and coated using 16 mil shims to entirely cover SS-1 (saturating the support substrate) and cured for 5 minutes at 130° C. The SGA-1 was then covered with an embossed low density polyethylene (LDPE) temporary release liner. The drug-in-adhesive layer of Example 1 was then laminated to the back of the SS-1. The laminates were allowed to equilibrate for 1 week prior to testing the drug flux.

### Example 3

The drug-in-adhesive layer was prepared by dispersing 0.3999 g ibuprofen and 0.2033 g K25 in 1.033 g ethyl acetate. To the dispersion, 0.1040 g 2-propanol was added to dissolve the ibuprofen and the K25. To this solution, 2.1760 g PSA-1 and 0.1110 g dipropylene glycol/oleic acid (2:1) blend was added. The mixture was mixed overnight in a vial and coated onto SCOTCHPAK® 1022 using a 15 mil applicator bar. The laminate was dried for 5 minutes at ambient conditions followed by 5 minutes at 92° C. The adhesive was laminated to SCOTCHPAK® 8038. The resulting coat weight was approximately 9.97 mg/cm² with an ibuprofen content of 2.0 mg/cm² (0.495 cm² patch-0.99 mg ibuprofen available).

### Example 4

To produce the skin-contact layer, equal parts A and B of SGA-1 were mixed and coated using 16 mil shims to entirely cover SS-1 (saturating the support substrate) and cured for 5 minutes at 130° C. The SGA-1 was then covered with an embossed LDPE as a temporary release liner. The drug-in-adhesive layer of Example 3 was then laminated to the back of the SS-1. The laminates were allowed to equilibrate for 1 week prior to testing the drug flux.

### Flux Experiments for Examples 1-4

Patches having a surface area of 0.495 cm² were fluxed through heat separated epidermis from full-thickness human cadaver skin. The receptor fluid was phosphate buffered saline pH 7.4 and the study was conducted at 32° C. Samples of the receptor fluid were taken at 1, 3, 7, 15, and 24 hours with full replacement using fresh phosphate buffered saline. Samples were analyzed for ibuprofen concentration using an appropriate UPLC method. Each formulation example was tested in triplicate, Benchmark 1 was fluxed with an application of 20 mg of product (1 mg ibuprofen available).

The data (FIG. 2 and Table 1) show that the incorporation of SGA-1 and SS-1 (Examples 2 and 4) increases the flux profile over that seen with the drug-loaded matrix alone (Examples 1 and 3). Table 1 shows that the cumulative release for both the drug-loaded matrix Examples 1 and 3 was at least 10 times higher than the cumulative release for the benchmark. The cumulative release for Example 2 was 11 times higher than the benchmark and the cumulative release for Example 4 was almost 20 times higher than the benchmark.

TABLE 1
Cumulative release (μg/cm²) and percent released (%) at 24 hours for
Examples 1-4 and Benchmark 1.

| | Cumulative | Percent |
|---|---|---|
| | Release | Released |
| Example | (μg/cm²) | (%) |
| Benchmark 1 | 24.6 | 1.2 |
| 1 | 251.8 | 12.1 |
| 2 | 305.9 | 14.7 |
| 3 | 330.3 | 16.6 |
| 4 | 498.2 | 25.0 |

### Example 5

The drug-in-adhesive layer was prepared by dispersing 1.0064 g ibuprofen and 1.0317 g VA64 in 2.0289 g ethyl acetate. To the dispersion, 1.0182 g 2-propanol was added to dissolve the ibuprofen and the VA64. To this solution, 13.7585 g PSA-2 and 0.9993 g oleic acid were added. To this, 0.3855 g ethyl acetate was added to adjust the solids content to approximately 50 wt. %. The mixture was mixed overnight in a vial and coated onto SCOTCHPAK® 1022 using a 15 mil applicator bar. The laminate was dried for 5 minutes at ambient conditions followed by 5 minutes at 92° C. The adhesive was laminated to SCOTCHPAK® 8038. The resulting coat weight was approximately 10.29 mg/cm² with an ibuprofen content of 1.02 mg/cm² (0.495 cm² patch-0.507 mg ibuprofen available).

### Example 6

To produce the skin-contact layer, equal parts A and B of SGA-2 were mixed and coated to entirely cover SS-2 using 16 mil shims and cured for 4 minutes at 130° C. The SGA-2 was then covered with an LDPE temporary release liner. The drug-in-adhesive layer of Example 5 was then laminated to the back of the SS-2. The laminates were allowed to equilibrate for 2 weeks prior to testing the drug flux.

### Example 7

To produce the skin-contact layer, equal parts A and B of SGA-2 were mixed and coated to entirely cover onto SS-3 using 16 mil shims and cured for 4 minutes at 130° C. The SGA-2 was then covered with an LDPE temporary release liner. The drug-in-adhesive layer of Example 5 was then laminated to the back of the SS-3. The laminates were allowed to equilibrate for 2 weeks prior to testing the drug flux.

### Example 8

To produce the skin-contact layer, equal parts A and B of SGA-2 were mixed and coated to entirely cover SS-1 using 16 mil shims and cured for 4 minutes at 130° C. After curing, a second coat of SGA-2 was applied and cured in the same fashion. The SGA-2 was then covered with an LDPE temporary release liner. The drug-in-adhesive layer of Example 5 was then laminated to the back of the SS-1. The laminates were allowed to equilibrate for 2 weeks prior to testing the drug flux.

### Example 9

To produce the skin-contact layer, 4.761 g SGA-2 Part A, 4.764 g SGA-2 Part B and 0.498 g isopropyl tetradecanoate were mixed and coated to entirely cover SS-2 using 16 mil shims and cured for 4 minutes at 130° C. The SGA-2 was then covered with an LDPE temporary release liner. The drug-in-adhesive layer of Example 5 was then laminated to the back of the SS-2. The laminates were allowed to equilibrate for 2 weeks prior to testing the drug flux.

### Example 10

To produce the skin-contact layer, 4.505 g SGA-2 Part A, 4.502 g SGA-2 Part B and 0.997 g isopropyl tetradecanoate were mixed and coated to entirely cover SS-2 using 16 mil shims and cured for 4 minutes at 130° C. The SGA-2 was then covered with an LDPE temporary release liner. The drug-in-adhesive layer of Example 5 was then laminated to the back of the SS-2. The laminates were allowed to equilibrate for 2 weeks prior to testing the drug flux.

### Example 11

To produce the skin-contact layer, equal parts A and B of SGA-1 were mixed and coated to entirely cover SS-1 using 16 mil shims and cured for 4 minutes at 130° C. The SGA-1 was then covered with an LDPE temporary release liner. The drug-in-adhesive layer of Example 5 was then laminated to the back of the SS-1. The laminates were allowed to equilibrate for 2 weeks prior to testing the drug flux.

### Example 12

To produce the skin-contact layer, equal parts A and B of SGA-1 were mixed and coated to form a pattern onto SS-1 using 16 mil shims and cured for 4 minutes at 130° C. The SGA-1 was then covered with an LDPE temporary release liner. The drug-in-adhesive layer of Example 5 was then laminated to the back of the SS-1. The laminates were allowed to equilibrate for 2 weeks prior to testing the drug flux.

Flux Experiments for Examples 5-12

Patches having a surface area of 0.495 cm² were fluxed through heat separated epidermis from full-thickness human cadaver skin. The receptor fluid was phosphate buffered saline pH 7.4 and the study was conducted at 32° C. Samples of the receptor fluid were taken at 4, 8, 12, 24, 36, 48, 60, and 72 hours with full replacement using fresh phosphate buffered saline. Samples were analyzed for ibuprofen concentration using an appropriate UPLC method. Each formulation example was tested in triplicate,

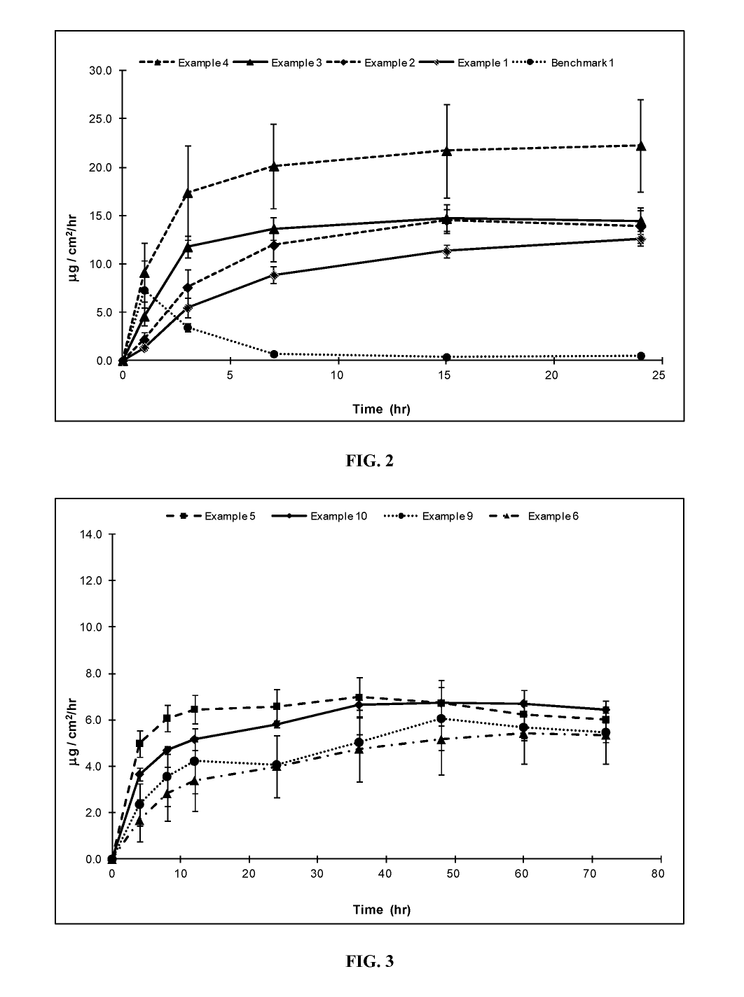

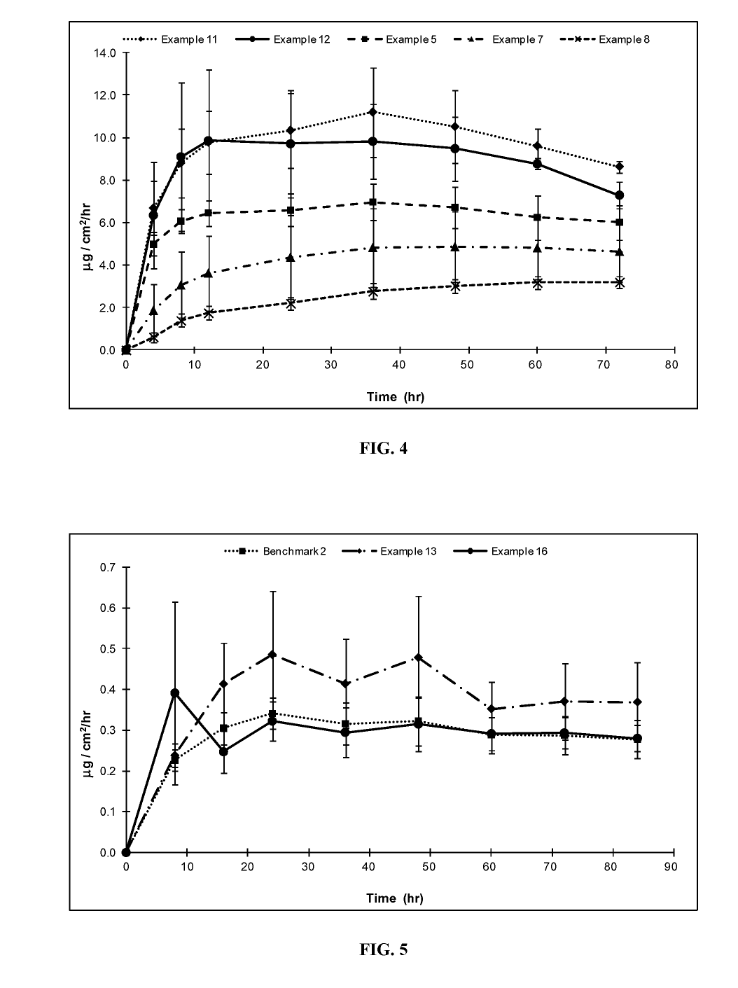

The data (FIGS. 3-4 and Table 2) show that the incorporation of SGA-2 and SS-2 (Examples 6, 9 and 10) decreases the flux profile over that seen with the drug-loaded matrix alone (Example 5). However, the incorporation of an enhancer, isopropyl tetradecanoate in Examples 9 and 10 into the SGA-2 increased the flux over that without the enhancer. However, the incorporation of SGA-1 and SS-1 (Examples 11-12) improved the flux over the drug loaded matrix alone (Example 5) as shown in FIG. 4. Example 8 shows the lowest flux-about 187 μg/cm² or 18% ibuprofen released after 72 hours; however, this construction has a double layer of SGA-2, so both the thickness and the selection of SGA account for the low flux. The data demonstrate non-limiting examples of how the current construction can be used to tune or customize the release rate to meet the desired therapeutic window of the active. Examples 11 and 12 exhibit the highest flux out of examples 5-12 with about 69 and 63%, respectively, released after 72 hours. Example 5 exhibited the third highest flux, with about 460 μg/cm² or 45% released. Examples 11 and 12 released about 50% more ibuprofen than that released in Example 5.

TABLE 2
Cumulative release (μg/cm²) and percent released (%) at 72 hours for
Examples 5-12.

| | Cumulative | Percent |
|---|---|---|
| | Release | Released |
| Example | (μg/cm²) | (%) |
| 5 | 460.3 | 45.0 |
| 6 | 327.4 | 32.0 |
| 7 | 316.3 | 30.9 |
| 8 | 187.1 | 18.3 |
| 9 | 356.1 | 34.8 |
| 10 | 441.8 | 43.2 |
| 11 | 704.9 | 68.9 |
| 12 | 641.9 | 62.7 |

### Example 13

The drug-in-adhesive layer was prepared by dispersing 0.1546 g estradiol and 0.7767 g VA64 in 0.9262 g ethyl acetate. To the dispersion, 0.4662 g 2-propanol was added to dissolve the estradiol and VA64. To this solution, 14.7478 g PSA-2, 0.9014 g oleic acid and 0.5992 g dipropylene glycol were added. To this, 1.4728 g ethyl acetate was added to adjust the solids content to approximately 50 wt. %. The mixture was mixed overnight in a vial and coated onto SCOTCHPAK® 1022 using a 15 mil applicator bar. The laminate was dried for 5 minutes at ambient conditions followed by 5 minutes at 92° C. The adhesive was laminated to SCOTCHPAK® 8038. The resulting coat weight was approximately 10.33 mg/cm² with an estradiol content of 0.154 mg/cm² (0.495 cm² patch-0.076 mg estradiol available).

### Example 14

To produce the skin-contact layer, equal parts A and B of SGA-2 were mixed and coated to entirely cover SS-2 using 16 mil shims and cured for 4 minutes at 130° C. The SGA-2 was then covered with an LDPE temporary release liner. The drug-in-adhesive layer of Example 13 was then laminated to the back of the SS-2. The laminates were allowed to equilibrate for 2 weeks prior to testing the drug flux.

### Example 15

To produce the skin-contact layer, equal parts A and B of SGA-2 were mixed and coated to entirely cover SS-3 using 16 mil shims and cured for 4 minutes at 130° C. The SGA-2 was then covered with an LDPE temporary release liner. The drug-in-adhesive layer of Example 13 was then laminated to the back of the SS-3. The laminates were allowed to equilibrate for 2 weeks prior to testing the drug flux.

### Example 16

To produce the skin-contact layer, equal parts A and B of SGA-2 were mixed and coated to entirely cover SS-1 using 16 mil shims and cured for 4 minutes at 130° C. After curing, a second coat of SGA-2 was applied and cured in the same fashion. The SGA-2 was then covered with an LDPE temporary release liner. The drug-in-adhesive layer of Example 13 was then laminated to the back of the SS-1. The laminates were allowed to equilibrate for 2 weeks prior to testing the drug flux.

### Example 17

To produce the skin-contact layer, 4.505 g SGA-2 Part A, 4.502 g SGA-2 Part B and 0.997 g isopropyl tetradecanoate were mixed and coated to entirely cover SS-2 using 16 mil shims and cured for 4 minutes at 130° C. The SGA-2 was then covered with an LDPE temporary release liner. The drug-in-adhesive layer of Example 13 was then laminated to the back of the SS-2. The laminates were allowed to equilibrate for 2 weeks prior to testing the drug flux.

### Example 18

To produce the skin-contact layer, equal parts A and B of SGA-1 were mixed and coated to entirely cover SS-1 using 16 mil shims and cured for 4 minutes at 130° C. The SGA-1 was then covered with an LDPE temporary release liner. The drug-in-adhesive layer of Example 13 was then laminated to the back of the SS-1. The laminates were allowed to equilibrate for 2 weeks prior to testing the drug flux.

### Example 19

To produce the skin-contact layer, equal parts A and B of SGA-1 were mixed and coated to form a pattern onto SS-1 using 16 mil shims and cured for 4 minutes at 130° C. The SGA-1 was then covered with an LDPE temporary release liner. The drug-in-adhesive layer of Example 13 was then laminated to the back of the SS-1. The laminates were allowed to equilibrate for 2 weeks prior to testing the drug flux.

Flux Experiments for Examples 13-19

Patches having a surface area of 0.495 cm² were fluxed through heat separated epidermis from full-thickness human cadaver skin. The receptor fluid was phosphate buffered saline pH 7.4 and the study was conducted at 32° C. Samples of the receptor fluid were taken 8, 16, 24, 36, 48, 60, 72 and 84 hours with full replacement using fresh phosphate buffered saline. Samples were analyzed for estradiol concentration using an appropriate UPLC method. Each formulation example was tested in triplicate, Benchmark 2 was used as a commercial benchmark for these examples.

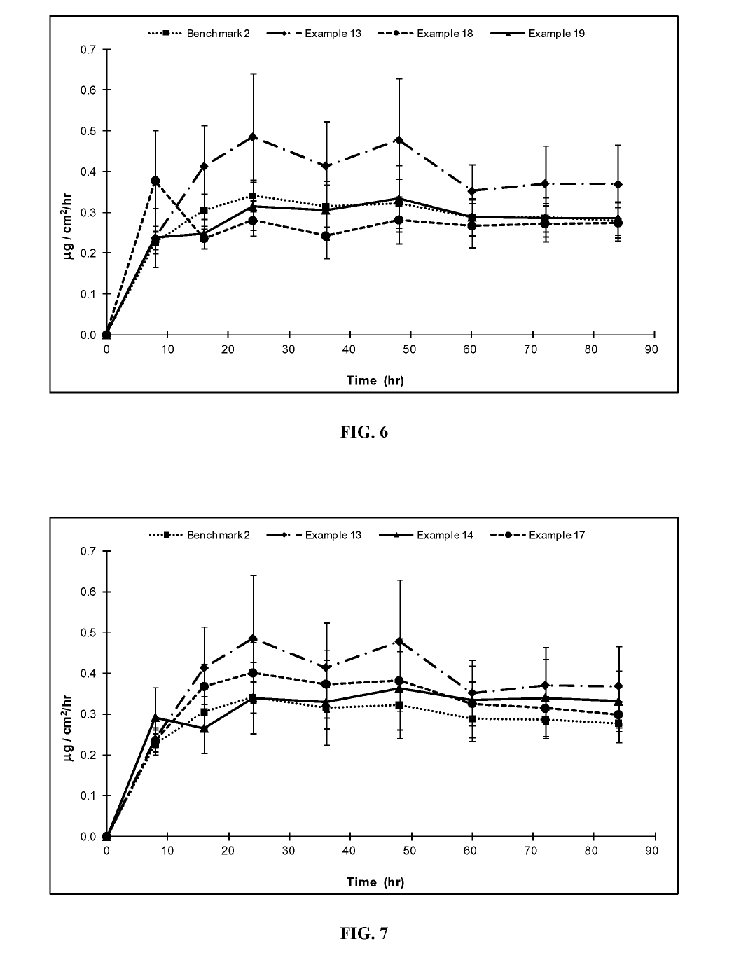

The data (FIGS. 5-8 and Table 3) show that the incorporation of SGA and SS with a drug-loaded matrix does not greatly change the flux profile for this particular API compared to the drug-loaded matrix alone. In all cases (Examples 13-19), the flux profile is similar to that of Benchmark 2.

TABLE 3
Cumulative release (μg/cm²) and percent released (%) at 84 hours for
Examples 13-19.

| | Cumulative | Percent |
|---|---|---|
| | Release | Released |
| Example | (μg/cm²) | (%) |
| Benchmark 2 | 24.9 | 15.9 |
| 13 | 32.9 | 21.4 |
| 14 | 27.6 | 17.9 |
| 15 | 27.9 | 18.1 |
| 16 | 25.4 | 16.5 |
| 17 | 28.4 | 18.4 |
| 18 | 23.2 | 15.1 |
| 19 | 24.4 | 15.9 |

### Example 20

The drug-in-adhesive layer was prepared by dispersing 1.0019 g ibuprofen and 1.0278 g VA64 in 1.9980 g ethyl acetate. To the dispersion, 0.9924 g 2-propanol was added to dissolve the ibuprofen and the VA64. To this solution, 14.1132 g PSA-2 and 0.9992 g oleic acid were added. The mixture was mixed overnight in a vial and coated onto SCOTCHPAK® 1022 using a 15 mil applicator bar. The laminate was dried for 5 minutes at ambient conditions followed by 5 minutes at 92° C. The adhesive was laminated to SCOTCHPAK® 8038. The resulting coat weight was approximately 9.46 mg/cm² with an ibuprofen content of 0.95 mg/cm² (0.495 cm² patch-0.468 mg ibuprofen available).

### Example 21

To produce the skin-contact layer, equal parts A and B of SGA-1 were mixed and coated to entirely cover SS-1 using 16 mil shims and cured for 5 minutes at 130° C. The SGA-1 was then covered with an LDPE temporary release liner. The drug-in-adhesive layer of Example 20 was then laminated to the back of the SS-1 using a 20 lb roller. The laminates were allowed to equilibrate for a minimum of 2 weeks prior to testing the drug flux.

### Example 22

To produce the skin-contact layer, equal parts A and B of SGA-1 were mixed and coated to entirely cover SS-2 using 16 mil shims and cured for 5 minutes at 130° C. The SGA-1 was then covered with an LDPE temporary release liner. The drug-in-adhesive layer of Example 20 was then laminated to the back of the SS-2 using a 20 lb roller. The laminates were allowed to equilibrate for a minimum of 2 weeks prior to testing the drug flux.

### Example 23

This example was prepared according to the methods described in WO2011/022199 A2. To produce the skin-contact layer, equal parts A and B of SGA-1 were mixed and coated to entirely cover an LDPE temporary release liner using 10 mil shims and cured for 15 minutes at 80° C. The drug-in-adhesive layer of Example 20 was then laminated to the SGA-1 using a 20 lb roller. The 20 lb roller did not provide enough pressure to adhere the SGA-1 to the drug-in-adhesive layer. The laminate was pressed using a hydraulic press with ˜500 psi pressure. The pressure required to achieve sufficient adhesion between the two layers resulted in deformation of both adhesive layers as exhibited by SGA being squeezed out the sides of the laminate. The defects introduced to the drug-in-adhesive layer during the pressing process by any component that is not impeccably smooth (e.g. embossed temporary release liners, defects in coating hardware, etc.) can also create nucleation sites resulting in uncontrolled crystallization of the drug.

Even with the increased pressure that provided sufficient adhesion between the two layers, the LDPE temporary release liner proved difficult to remove. Only approximately 50% of the SGA 1 transferred to the drug-in-adhesive layer, which would result in 50% wasted product that did not transfer. The inability to transfer the SGA is inherent in this material due to the low cohesive strength, low surface adhesion. and relatively high affinity of the SGA to the substrate it is cured upon.

Typically the SGA material is cured at temperatures of approximately 130° C. The LDPE temporary release liner cannot withstand this curing temperature and will deform. To cure the SGA onto the LDPE, a lower temperature is required. SGA-1 was cured at 80 and 130° C. using an Alpha Technologies MDR2000 to determine the difference in cure time at these temperatures (Table 4). The t50 time represents the time it takes to reach 50% of the final consistency of the cured material as measured by torque whereas the t90 is the time required to reach 90% of the maximum torque. The t90 time is typically reported as the time required to fully cure the material. At the lower cure temperature of 80° C., it takes about 10 times longer to cure the SGA onto the LDPE. Specifically, the cure time at 130° C. was 1 minute 33 seconds, and the cure time at 80° C. was 15 minutes 25 seconds.

TABLE 4
Cure time for SGA-1 at 80 and 130° C.

| Temp. | t50 | t90 |
|---|---|---|
| (° C.) | (m:s) | (m:s) |
| 80 | 5:40 | 15:25 |
| 130 | 0:35 | 1:33 |

The methods and systems described in WO2011/022199 A2 would be impractical to implement in a commercial setting, as those methods and systems require significantly longer curing times, increase the amount of pressure required to transfer the SGA onto the LDPE, and result in low product yields of about 50% or less, causing a large amount of product waste. The methods and systems according to the present disclosure result in product yields of 100%. The yield in examples 1-19 where the silicone gel adhesive layer was coated onto the front of the support substrate and the drug-in-adhesive layer was laminated to the back of the support substrate was 100%.

Flux Experiments for Examples 20-23

Patches having a surface area of 0.495 cm² were fluxed through heat separated epidermis from full-thickness human cadaver skin. The receptor fluid was phosphate buffered saline pH 7.4 and the study was conducted at 32° C. Samples of the receptor fluid were taken 4, 8, 12, 24, 36, 48, 60 and 72 hours with full replacement using fresh phosphate buffered saline. Samples were analyzed for ibuprofen concentration using an appropriate UPLC method. Each formulation example was tested in triplicate,

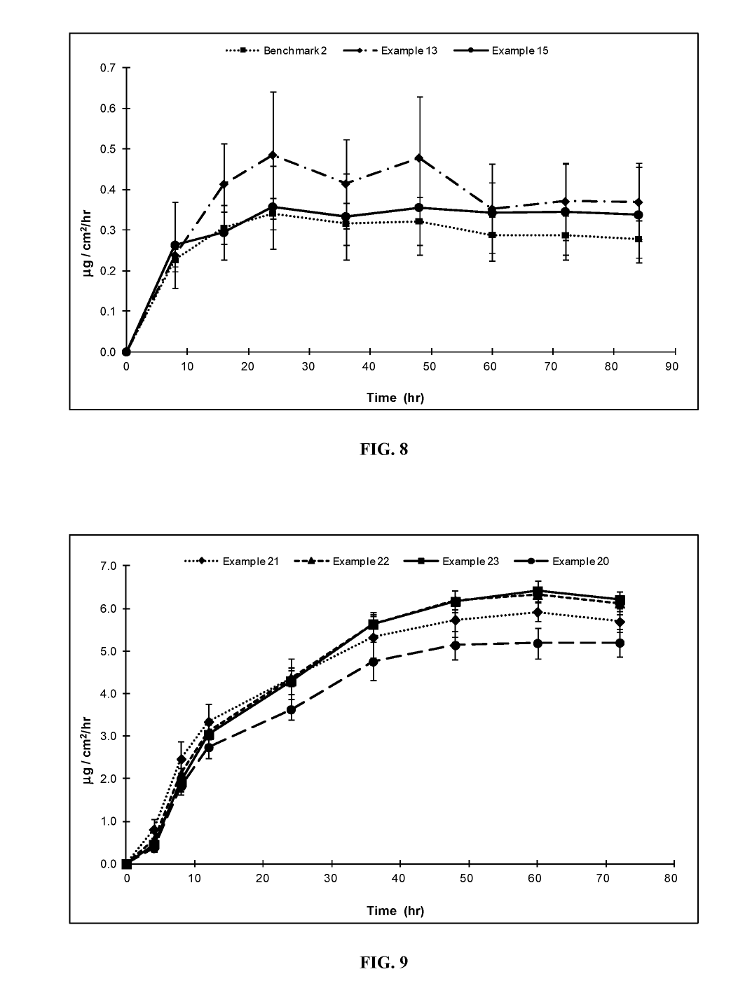

It was anticipated that the addition of a support substrate between the drug-in-adhesive and SGA layers would reduce the release/flux of the drug. Surprisingly, these data (FIG. 8 and Table 5) show that the addition of a support substrate (SS) between the drug-loaded matrix and the SGA does not significantly decrease the flux profile as compared to SGA with a drug-loaded matrix and no SS (construct described in WO2011/022199 A2). The incorporation of the SS layer in the construction described herein, does however, greatly increase the ease of production.

TABLE 5
Percent released (%) at 24 hours for Examples 20-23.

| | Percent |
|---|---|
| | Released |
| Example | (%) |
| 20 | 30.4 |
| 21 | 36.3 |
| 22 | 43.0 |
| 23 | 36.3 |

While the present disclosure is susceptible to various modifications and alternative forms, specific embodiments have been shown by way of example in the examples and described in detail hereby. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

What is claimed is:

1. A multi-layer delivery system, comprising:
an occlusive or non-occlusive backing substrate;
a drug-loaded layer including a therapeutic concentration of at least one or a combination of cosmetic and/or pharmaceutical active ingredients;
a permeable or semi-permeable support substrate;
a non-drug loaded silicone gel adhesive layer obtained by (i) reacting a gel producing composition comprising (A) at least one alkenyl-substituted polydiorganosiloxane, (B) at least one organosiloxane containing silicone-bonded hydrogen atoms, and (C) at least one catalyst for the reaction of the SiH groups with the Si-alkenyl groups; and (ii) curing the product of (i); the non-drug loaded silicone gel adhesive layer is then cured onto the permeable or semi-permeable support substrate; and
a temporary release liner configured to protect the cured silicone gel adhesive layer.

2. The system of claim 1, wherein the system is a multi-layer drug delivery system wherein the drug-loaded layer is positioned between and in contact with the backing substrate and the support substrate and the non-drug loaded silicone gel adhesive layer is positioned between and in contact with the support substrate and the temporary release liner.

3. The system of claim 1, wherein the support substrate is configured to withstand curing of the non-drug loaded silicone gel adhesive layer at elevated temperatures of up to about 150° C.

4. The system of claim 1, wherein the support substrate is a permeable, semi-permeable, or microporous material onto which the non-drug loaded silicone gel adhesive layer is coated and cured, wherein the support substrate optionally has a thickness of between about 0.010 mm and about 1 mm, the support substrate being rate controlling or not rate controlling.

5. The system of claim 1, wherein the system is configured to deliver between about 0.1 to about 80 percent of the at least one or a combination of cosmetic and/or pharmaceutical active ingredients to the skin, wherein the backing substrate is an occlusive or non-occlusive film composed of silicone, plastics, polyesters, polyurethanes, high or low density polyethylene, ethyl vinyl acetate, polyethyleneterephatlate, polycarbonate, or any combination thereof.

6. The system of claim 1, wherein the drug-loaded layer is a non-curing silicone pressure sensitive adhesive or wherein the drug-loaded layer is a silicone-acrylic hybrid, polyisobutylene and derivatives thereof, acrylics, natural rubbers, natural and synthetic polyisoprene, polybutylene and polyisobutylene, styrene/butadiene polymers, styrene-isoprene-styrene block polymers, hydrocarbon polymers such as butyl rubber, halogen polyvinylchloride, polyvinylidene chloride, polyvinylpyrrolidone and polychlorodiene, viscoelastic pressure sensitive adhesive that is an acrylic adhesive or a synthetic rubber pressure sensitive adhesive, or any combination thereof.

7. The system of claim 1, wherein the at least one or a combination of cosmetic and/or pharmaceutical active ingredients is a non-steroidal anti-inflammatory drug, retinoid, antifungal, steroid, vitamin, or traditional Chinese medicine (TCM), wherein the drug-loaded layer optionally includes between at least about 0.01 and about 80 percent by weight of the active ingredient.

8. The system of claim 1, wherein the drug-loaded layer and/or the non-drug loaded silicone gel adhesive layer is free of penetration enhancers and/or wherein the drug-loaded layer is free of stabilizers.

9. The system of claim 1, wherein the drug-loaded layer and/or the non-drug loaded silicone gel adhesive layer include between about 0.5 to about 25 percent by weight of one or more penetration enhancers and/or wherein the drug-loaded layer includes between about 0.01 to about 25 percent by weight of one or more stabilizers.

10. The system of claim 1, wherein the non-drug loaded silicone gel adhesive layer is a silicone resin-reinforced silicone gel adhesive that includes between about 2 to about 45 percent by weight of at least one hydroxyl substituted siloxane resin.

11. The system of claim 1, wherein the non-drug loaded silicone gel adhesive layer is coated and cured onto the support substrate wherein the support substrate is fully covered with a silicone gel adhesive or wherein the non-drug loaded silicone gel adhesive layer is coated and cured into a pattern onto the support substrate.

12. The system of claim 1, wherein the non-drug loaded silicone gel adhesive layer has a thickness of from about 0.025 mm to about 2 mm, wherein the non-drug loaded silicone gel adhesive layer saturates the support substrate.

13. The system of claim 1, wherein the non-drug loaded silicone gel adhesive layer has a thickness of from about 0.025 mm to about 2 mm, wherein the non-drug loaded silicone gel adhesive layer does not saturate the support substrate.

14. A process for the manufacture of a multi-layer drug delivery system comprising:
laminating a drug-loaded layer onto a backing substrate;
laminating the drug-loaded layer together with the backing substrate onto a first side of a support substrate; and
laminating a non-drug loaded silicone gel adhesive layer onto a second side of the support substrate and applying a temporary release liner onto the non-drug loaded silicone gel adhesive layer.

15. A process for the manufacture of a multi-layer drug delivery system comprising:
laminating a drug-loaded layer onto a backing substrate;
coating and curing a non-drug loaded silicone gel adhesive layer onto a first side of a support substrate and covering said non-drug loaded silicone gel adhesive layer with a temporary release liner; and
laminating the drug-loaded layer together with the backing substrate onto a second side of the support substrate.
